(19) United States
(12) Patent Application Publication
(10) Pub. No.: US 2002/0169209 A1
(43) Pub. Date: Nov. 14, 2002

(54) Potentiation of therapeutic effects of fatty acids

(76) Inventor: David Frederick Horrobin, Stirling (GB)

Correspondence Address: JACOBSON, PRICE, HOLMAN & STERN, PROFESSIONAL LIMITED LIABILITY COMPANY, 400 Seventh Street, N.W., Washington, DC, 20004, US

(21) Appl. No.: 10/134,501

(22) Filed: Apr. 30, 2002

(30) Foreign Application Priority Data
May 5, 2001 (GB) 0111282.0

(51) Int. Cl. A61K 31/202

(52) U.S. Cl. 514/560

(57) ABSTRACT

The oral administration of an essential fatty acid, preferably eicosapentaenoic acid, at a defined purity together with an inhibitor of COX-1 or COX-2 or LOX or one or more of the FACL enzymes gives improved therapeutic results over administration of the fatty acid alone.

Unsaturated fatty acids of the omega-6 and omega-3 series have many potential uses. The present inventor and other inventors have obtained numerous patents and filed many patent applications which deal with the therapeutic effects of unsaturated fatty acids in many different disorders including cancers, skin disorders, inflammatory disorders, menstrual cycle disorders, reproductive disorders, renal and urinary tract disorders, metabolic disorders including diabetes mellitus, osteoporosis, urolithiasis and other disorders of calcium metabolism, gastrointestinal disorders, respiratory system disorders, and central nervous system disorders including neurological and psychiatric disorders. Examples of granted patents which demonstrate that these fatty acids have a wide range of utility in many diseases are the following US cases: U.S. Pat. Nos. 4,826,877; 5,847,000; 5,457,130; 4,302,447; 4,681,896; 5,198,468; 5,922,345.

This specification concerns methods for improving the efficacy of treatments with unsaturated fatty acids.

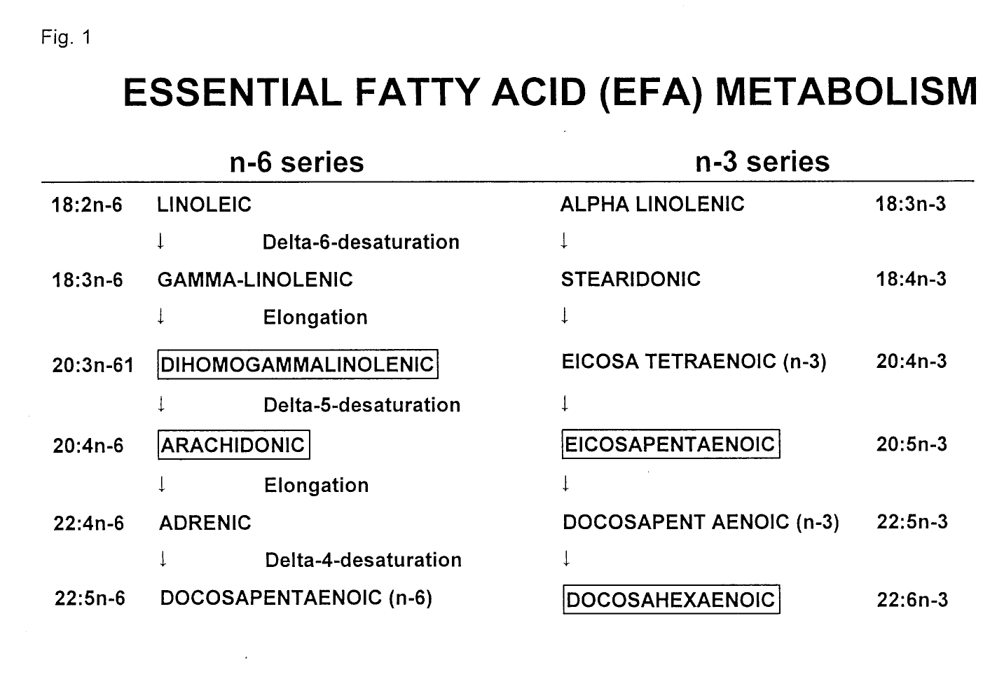

The pathways of metabolism of the unsaturated essential fatty acids (EFAs) are shown in FIG. 1. The EFAs are like vitamins in the sense that they are required for human and animal metabolism but cannot be synthesised de novo by the mammalian body. There are two sorts of EFAs: the n-6 (or omega-6) and the n-3 (or omega-3). The parent compounds linoleic acid (LA) of the n-6 series and alpha-linolenic acid (ALA) of the n-3 series are the main compounds found in the diet. However, to be useful to the body, these parent compounds must be converted to the so-called derived essential fatty acids shown in FIG. 1. These derived EFAs play key roles in the structures of all internal and external cell membranes. They are also released from these cell membranes following many different types of cell activation which convert phospholipases A₂, C and D to active forms and which directly or indirectly lead to release of the free acids from membrane phospholipids.

These free fatty acids then partake in many different signalling processes which modify many aspects of cellular function. The fatty acids which are of particular importance are three fatty acids which are good substrates for the cyclo-oxygenase (COX) group of enzymes, dihomogammalinolenic acid (DGLA), arachidonic acid (AA) and eicosapentaenoic acid (EPA) and another fatty acid, docosahexaenoic acid (DHA), which although a poor subject for COX is also an important component of membrane phospholipids. Gamma-linolenic acid (GLA), which is an effective precursor of DGLA and AA, and stearidonic acid (SA), which is an effective precursor of EPA, are also potentially important molecules.

There are two main types of COX. COX-1 is a constitutively expressed enzyme which continuously converts the relevant derived fatty acid to low to moderate levels of prostaglandins and related substances. COX-2 is an enzyme which is expressed in large amounts in most tissues when they are reacting to any form of change or stimulation. Thus COX-2 is expressed in large amounts whenever there is an inflammatory process of any sort, whenever cells proliferate abnormally as in cancer cells and the blood vessels supplying them, and in any situation where cells are dying or degenerating including neurodegenerative disorders like Parkinson's disease Alzheimer's disease, other forms of dementia, including vascular dementia, amyotrophic lateral sclerosis, Huntington's disease and other neurological disorders involving “triplet repeats” such as Friedreich's ataxia, spinocerebellar ataxia and myotonic dystrophy.

The free fatty acids released by phospholipases can also be converted to a range of other eicosanoids by a group of enzymes known as lipoxygenases (LOX). The products of the COX and LOX enzymes are believed to mediate many and perhaps most of the biological actions of the free fatty acids. The other major routes of disposal of the free fatty acids are oxidation and linkage to coenzyme A by a group of enzymes known as fatty acid coenzyme-A ligase (FACL) or alternatively as acyl-CoA synthetase (ACS). Linkage to CoA is a necessary step prior to the entry of fatty acids into any one of a large number of synthetic and degradative pathways.

There have been many proposals concerning the therapeutic uses of EFAs and derived EFAs, particularly GLA, DGLA and to some extent AA of the n-6 series, and EPA docosapentaenoic acid (DPA), DHA and to some extent SA of the n-3 series. Most of these proposals have assumed that a substantial part of the therapeutic effects of the EFAs and derived EFAs depend on their conversion to highly active metabolites by the COX and LOX groups of enzymes.

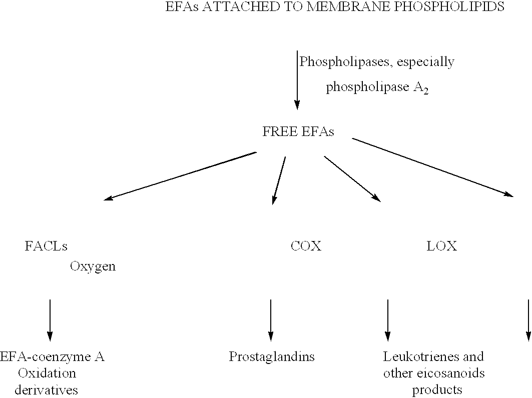

However, there is increasing evidence that many of the actions of the EFAs and derived EFAs are mediated not by the metabolites but by the fatty acids themselves. There appear to be many different mechanisms. As examples, some ion channels have binding sites for EFAs and their function can be modified, so regulating the movements of sodium, potassium, calcium and chloride channels. Or some protein kinases and other enzymes have allosteric binding sites for fatty acids which lead to their activation or inhibition. Or some genes may be directly regulated by the binding of fatty acids to DNA. Or some receptors, notably the various types of peroxisome proliferator activated receptors (PPAR) may be activated by fatty acids and lead to a wide range of changes in cellular function. Or some fatty acids may be able to cause cell death either by apoptosis (programmed cell death) or by other means. Interestingly some fatty acids, especially GLA, DGLA and EPA and to a lesser extent SA, AA and DHA seem to be able to kill malignant cells selectively without harming normal cells.

Fatty acids of different structures often interact with the same binding sites on enzymes, receptors, transport proteins and regulatory control sites. Different fatty acids may act as agonists, antagonists or have neutral effects at such sites. In the past it has been common for fatty acids with presumed therapeutic actions to be administered in the form of complex mixtures such as fish oils containing eicosapentaenoic acid and docosahexaenoic acid, or plant, algal, fungal or other microbial oils containing gamma-linolenic acid or arachidonic acid or stearidonic acid. These plant oils are often rich in linoleic acid. It has been assumed that the effect of the oil is that of the most biologically interesting fatty acid, though usually without any experimental evidence that this is the case. The present inventor has been investigating this and has found that the use of partially or fully purified fatty acids or fatty acid derivatives often produces therapeutic effects which are greater than expected on the basis of the known effects of the natural oils. Though not prior art, reference is made here to a paper by the present inventor: Horrobin, D F. A new category of psychotropic drugs: neuroactive lipids as exemplified by ethyl eicosapentaenoate. Progress in Drug Research, September 2002.

The present invention relates to pharmaceutical formulations in which an EFA or derived EFA is used with an enzyme inhibitor selected from an inhibitor of COX-1 or COX-2 or LOX or one or more of the FACL enzymes. There are provided pharmaceutical formulations for oral administration in which a fatty acid preparation containing more than 70% eicosapentaenoic acid or eicosapentaenoic acid derivative and less than 10% docosahexaenoic acid or docosahexaenoic acid derivative and less than 10% linoleic acid or linoleic acid derivative is combined in the same dosage form or same pack with an enzyme inhibitor selected from an inhibitor of COX-1 or COX-2 or LOX or one or more of the FACL enzymes.

The main fatty acid or derivative of the fatty acid preparation should have a purity level such that interference at the key points of biological action from other fatty acids or fatty acid derivatives is reduced. The fatty acid or derivative present in the fatty acid preparation used in the present formulations should be at least 70% pure and preferably at least 80% pure. It is especially preferred that the fatty acid or derivative is 90% or 95% pure. In particular, there are two essential fatty acids which are commonly found in oils and which can play a role in the interference of the actions of the therapeutic fatty acids: docosahexaenoic acid and linoleic acid. It is a requirement that the docosahexaenoic acid or derivative and the linoleic acid or derivative present is each less than 10%, preferably less than 5% and very preferably less than 1% of any preparation of a fatty acid or fatty acid derivative used in the formulations of the present invention.

Eicosapentaenoic acid, EPA, is the most important essential fatty acid used in the fatty acid preparation of the present formulations, but it can be replaced by or added to by preparations containing any one or more of gamma-linolenic acid (GLA), dihomogamma-linolenic acid (DGLA), arachidonic acid (AA) and stearidonic acid (SA). In each case, the preparations of these other fatty acids should contain low levels of docosahexaenoic acid and linoleic acid as described in the previous paragraph.

Derivatives of the essential fatty acid which may be used in the present invention include: salts such as sodium, potassium or lithium salts; esters such as ethyl esters and cholesterol esters; mono-, di- and triglycerides; amides; phospolipids; and any other derivatives able to raise the levels of the fatty acid in the blood or tissues.

The enzyme inhibitor is preferably a combined inhibitor of COX-1 and COX-2, or a selective COX-2 inhibitor, or an inhibitor of one of the LOX group of enzymes, or a combined inhibitor of both COX and LOX enzymes. Those of particular interest include inhibitors of 5-lipoxygenase, which inhibit both COX-1 and COX-2, or which inhibit COX-2 selectively. Examples of selective or relatively selective COX-2 inhibitors are celecoxib, rofecoxib parecoxib, valdecoxib, etoricoxib and several other “coxibs”, nabumetone, nimesulide, meloxicam, chromene and aroylnapthalene compounds reported in WO 9847890 and WO 9832732, and a range of compounds such as those described in G Dannhardt and S Laufer, Current Medicinal Chemistry 2000; 7: 1101-12. Examples of non-selective or relatively non-selective COX-1 and COX-2 inhibitors include salicylic acid derivatives such as aspirin, sodium salicylate and sulfasalazine, para-aminophenol derivatives such as acetaminophen, indole and indene acetic acids such as indomethacin and sulindac, heteroaryl acetic acids such as tolmetin and diclofenac, arylpropionic acids such as ibuprofen, naproxen and ketoprofen, fenamates such as mefenamic acid and enolic acids such as piroxicam and phenylbutazone.

Work has shown the administration of EFAs to have therapeutically beneficial results in the treatment of many diseases. The advantages of the present invention will be therefore widespread. Case studies follow, but the applications are expected to be diverse, based on the present and future knowledge of the uses of EFAs.

The formulations of the present invention are suited for the treatment of any form of cancer and cancer cachexia and the present invention further provides such treatment and the use of the combination of EFA or derived EFA with the above enzyme inhibitors in a method of manufacture of a medicament for the treatment of cancer or cancer cachexia.

The formulations are also suited for the treatment of any form of psychiatric disease including schizophrenia, schizoaffective disorders, schizotypy, depression, anxiety, bipolar disorder, mania, borderline personality disorder, alcoholism and attention deficit hyperactivity disorder or any other psychiatric illness and the present invention provides such treatment and the use of the combination of EFA or derived EFA with the above enzyme inhibitors in a method of manufacture of a medicament for the treatment of any such psychiatric disease.

The formulations may be used in the treatment of any form of neurological or neurodegenerative disease including Parkinson's disease, Alzheimer's disease, amyotrophic lateral sclerosis, Huntington's disease and other “triplet-repeat” diseases, stroke, multi-infarct and other forms of dementia, multiple sclerosis, chronic fatigue and epilepsy and the present invention provides such treatment and the use of the combination of EFA or derived EFA with the above enzyme inhibitors in a method of manufacture of a medicament for the treatment of any such neurological or neurodegenerative disease.

The formulations are suited for the treatment of any form of inflammatory disease including any form of arthritis, any form of inflammatory skin disease including psoriasis and eczema, asthma, any form of inflammatory gastrointestinal disease including ulcerative colitis and Crohn's disease, and any inflammatory conditions of any other organs including the kidneys, the reproductive system, the eyes and the brain and the present invention provides such treatment and the use of the combination of EFA or derived EFA with the above enzyme inhibitors in a method of manufacture of a medicament for the treatment of any such inflammatory disease.

The formulations may be used in the treatment of any form of cardiovascular or cerebrovascular disease and the present invention provides such treatment and the use of the combination of EFA or derived EFA with the above enzyme inhibitors in a method of manufacture of a medicament for the treatment of any cardiovascular or cerebrovascular disease.

The formulations may be used in the treatment of any form of respiratory disease, including asthma or chronic obstructive pulmonary disease, and the present invention provides such treatment and the use of the combination of EFA or derived EFA with the above enzyme inhibitors in a method of manufacture of a medicament for the treatment of any respiratory disease, including asthma or chronic obstructive pulmonary disease.

The formulations may be used in the treatment of any form of metabolic disease including diabetes, syndrome X, and any disturbance of calcium metabolism including osteoporosis, urolithiasis, or urinary tract stone formation and the present invention provides such treatment and the use of the combination of EFA or derived EFA with the above enzyme inhibitors in a method of manufacture of a medicament for the treatment of any such metabolic disease.

The formulations may be used in the treatment of any form of renal or urinary tract disease and the present invention provides such treatment and the use of the combination of EFA or derived EFA with the above enzyme inhibitors in a method of manufacture of a medicament for the treatment of any renal or urinary tract disease.

The formulations may be used in the treatment of any form of disease or disorder of the reproductive system or menstrual cycle, including breast pain, premenstrual syndrome, dysmenorrherea or endometriosis, and the present invention provides such treatment and the use of the combination of EFA or derived EFA with the above enzyme inhibitors in a method of manufacture of a medicament for the treatment of disease or disorder of the reproductive system or menstrual cycle, including breast pain, premenstrual syndrome, dysmenorrherea or endometriosis.

It is surprising that the therapeutic effects of EFAs and derived EFAs may be substantially enhanced by combining administration of the EFA with a drug which blocks the conversion of the EFA to its metabolites. Drugs which block the COX or LOX or FACL groups of enzymes may be of particular interest.

This is an unexpected proposal because it is generally believed that the COX and LOX enzymes inhibitors exert their therapeutic effects not by conserving fatty acids but by blocking their conversion to prostaglandins, leukotrienes and other eicosanoids. It was conventionally thought that the lowering of eicosanoid levels is the critical mechanism of action; and a Nobel Prize was awarded to Vane, Samuelsson and Bergstrom for proposing this, so it is clearly a mainstream concept. The last thing any skilled person would want to do therefore is to propose administering substrates for the COX and LOX enzymes at the same time as providing COX and LOX inhibitors. Suchran action would enhance the formation of the eicosanoids whose production the drug was designed to block. Indeed investigators have often proposed that the way to improve therapy in diseases which may respond to COX and LOX inhibitors is to reduce the levels of the relevant fatty acids by dietary or other means. Thus, for example, a major pharmaceutical company which is expert in the field of fatty acids and of prostaglandin synthesis is developing inhibitors of delta-6- and delta-5-desaturases as anti-inflammatory agents (M G Obukowicz et al, J Pharmacol Exp Ther 1998; 287: 157-166). The aim of these drugs is to reduce the levels of prostaglandin precursors such as arachidonic acid, dihomogammalinolenic acid and eicosapentaenoic acid. Since the COX and LOX inhibitors are anti-inflammatory agents also, this teaching points completely away from the idea that the therapeutic effects of COX and LOX inhibitors might actually be enhanced by increasing the levels of these fatty acids.

In contrast, the result of the administration of the formulations of the present invention is that the therapeutic effects of EFAs and of drugs which inhibit EFA metabolism by LOX, COX, or FACL enzyme will be dramatically enhanced by the co-administration of the fatty acid with the drug. This concept may be applied to any present or future LOX, COX or FACL inhibitors. Drugs of particular interest in this respect are compounds which inhibit COX-1 and COX-2, or COX-2 selectively, or LOX selectively or COX and LOX together. This is because these compounds are widely used and understood and are readily available for administration.

The invention provides for the co-administration, whether in a single or separate formulation of one or more EFA or derived EFAs, selected from GLA, DGLA, AA, SA and EPA, preferably eicosapentaenoic acid and/or gamma-linolenic acid which are well tolerated in high doses, together with one or more drugs which inhibit COX-1 or COX-2, one or more of the LOX enzymes or one or more of the FACL enzymes. Drugs of particular interest are ones which inhibit 5-lipoxygenase, which inhibit both COX-1 and COX-2, or which inhibit COX-2 selectively. The fatty acids may be used in doses from 5 mg to 50 g/day, preferably 100 mg to 20 g/day and very preferably from 500 mg to 10 g/day. They may be used in any appropriate form which will raise the levels of the fatty acids in bodily tissues. Appropriate forms may include free acids, salts, esters such as ethyl esters mono-, di-, and triglycerides, amides, cholesterol esters, phospholipids, and any other appropriate forms. The enzyme inhibitors may be used in the doses which have been found to be safe and effective for each individual drug. Other conventional pharmaceutical ingredients may be present. The blue-green algae spirulina is not included as a therapeutic agent which may be used in the present formulations because in its native form the oil is likely to contain too much linoleic acid and not enough of any of the target fatty acids. The present formulations are intended for oral administration. Topical application routes are not included.

The aim of such combined administration is to elevate the levels of the fatty acids in cells by providing the fatty acid or its precursor, together with a drug which blocks the metabolism of the fatty acid by one or other of its metabolic routes. The invention may be illustrated by the following examples:

### EXAMPLES

An 80-year-old woman was diagnosed with inoperable colon cancer which had metastasised to the liver. She was given only about two months to live. In an effort to control the growth of the tumour she was given 2 g/day of ethyl-EPA together with 1 g/day of AA in triglyceride form. There was a modest beneficial effect and she was still alive after four months. But the cancer was still clearly growing, albeit at a reduced rate. She was then given in addition 200 mg bd (that is, twice a day, morning and evening) of celecoxib a selective COX-2 inhibitor. The cancer appeared to stop growing, she became healthier and was still alive 12 months after first initiating treatment with the fatty acids. She alter died, but the combination treatment had prolonged her life.

A 45-year-old woman had been seriously depressed for over 10 years and had failed to respond to treatment with several different antidepressants of various classes. Because there is evidence of elevated formation of prostaglandins in depression, she was given a combined COX-1 and COX-2 inhibitor, ibuprofen. She thought this might have had a marginal effect but this was not apparent to observers. The ibuprofen was stopped and she was treated with ethyl-eicosapentaenoate at a dose of 1 g/day. This seemed to have a modest effect but she remained far from well. However, when ibuprofen was added to the EPA there was a dramatic improvement and she has remained well for four months. The combination of ibuprofen, a COX-1 and COX-2 inhibitor, with EPA had a substantial beneficial effect which was achieved by neither drug alone.

A 52-year-old man had suffered from rheumatoid arthritis for many years. He had usually been treated with standard non-steroidal anti-inflammatory drugs (NSAIDs). While these had produced some relief of pain the effects were modest and he had major gastric side effects. Six months previously he had switched to one of the new selective COX-2 inhibitors, celecoxib. This had produced no better therapeutic effect on the arthritis but had substantially improved the gastric side effects. In addition to the celecoxib, he then took 2 g/day of GLA in the form of an enriched microbial oil. After about 3 months his arthritis began to improve substantially, the swelling of his joints subsided and for the first time for many years he felt he was getting on top of the disease.

An 81-year-old woman had become very forgetful and her family was concerned that she might be developing Alzheimer's disease (AD). Because there is evidence that the NSAID indomethacin may be able to slow down the progression of AD, her son, who was a doctor, prescribed indomethacin. Over six months or so this may have had a slight effect but there was no dramatic change. Because of animal work on the beneficial effects of AA in the aging rat brain, 800 mg of AA in triglyceride form was added to the treatment regime. Over the following 12 weeks the patient experienced a substantial renewal of energy, became more aware of what was going on around her and showed a considerable improvement in her memory for daily events.

A 61-year-old man developed a non-Hodgkin's lymphoma with multiple enlarged lymph nodes in the neck, the inguinal regions and the abdomen. For various reasons he did not wish to start standard chemotherapy and so was instead administered 8 g/day of eicosapentaenoic acid as the pure ethyl ester. This was done because of experimental evidence indicating that eicosapentaenoic acid could induce apoptosis in tumour cells without harming normal tissue. This produced some reduction in tumour size although the palpable masses remained substantial. After four weeks he was therefore treated in addition to the eicosapentaenoic acid with a high dose of celecoxib, 200 mg four times per day. On the fourth day of the regime there was a dramatic effect with disappearance of palpable tumours within 48 hours. The patient was temporarily very ill with malaise, fever and a skin rash for about 72 hours probably due to the rapid tumour lysis. The disappearance of the tumours was later confirmed by CT scan. Thus in this case the fatty acid alone had only a modest effect but the addition of the COX-2 inhibitor produced a dramatic response.

These five case histories illustrate the benefits of combining in therapy an EFA together with an inhibitor of COX-1, COX-2 or the LOX enzymes.

### Example Formulations

1. Formulations of hard or soft gelatin capsules in which each capsule contains between 100 mg and 1000 mg of eicosapentaenoic acid in ethyl ester or triglyceride form, where the EPA preparation contains at least 70% eicosapentaenoic acid derivative, less than 10% docosahexaenoic acid or derivative, and less than 10% linoleic acid or derivative, together with an inhibitor of COX-1 or COX-2 or LOX or a drug which has multiple inhibitory effects on those enzymes. The fatty acid dose should be adjusted to provide between 50 mg and 10,000 mg daily and the daily dose of fatty acid should also provide for an appropriate daily dose of the COX or LOX inhibitor.

2. Combination packs in which the eicosapentaenoic acid of example formulation 1 is provided in a hard or soft gelatin capsule while the COX or LOX inhibitor is provided in a tablet, capsule or other appropriate dosage form, the two types of dosage form being provided in the same overall pack with a set of instructions for their combined use.

3. Formulations as in 1 and 2 where the eicosapentaenoic acid preparation contains more than 90% eicosapentaenoic acid and where the levels of linoleic acid or of docosahexaenoic acid are each below 5%, and preferably below 1%.

4. As in example formulations 1 to 3 where the EPA is replaced by or supplemented with a fatty acid preparation selected from GLA, DGLA, AA and SA.

5. As in example formulations 1 to 4 where the drug is a non-steroidal anti-inflammatory agent with a dual action on COX-1 and COX-2 such as aspirin, ibuprofen, indomethacin or any one of the many drugs in this class.

6. As in example formulations 1 to 4 where the drug is a selective COX-2 inhibitor such as celecoxib, rofecoxib or any other selective inhibitor.

7. As in example formulations 1 to 4 where the drug is a compound which has LOX inhibitory activity with or without COX inhibitory activity.

8. As in example formulations 1 to 4 where the drug is a compound which inhibits fatty acid coenzyme-A ligases (FACL).

9. As in example formulations 1 to 4 where two or more drugs with COX-, LOX- or FACL-inhibiting activity are combined.

1. Pharmaceutical formulations for oral administration in which a fatty acid preparation containing more than 70% eicosapentaenoic acid (EPA) or eicosapentaenoic acid derivative and less than 10% docosahexaenoic acid or a docosahexaenoic acid derivative and less than 10% linoleic acid or a linoleic acid derivative is combined in the same dosage form or same pack with an enzyme inhibitor selected from the group consisting of an inhibitor of COX-1 and/or COX-2, an inhibitor of LOX and an inhibitor of one or more of the FACL enzymes.

2. Pharmaceutical formulations according to claim 1 in which the fatty acid preparation contains more than 80% eicosapentaenoic acid or eicosapentaenoic acid derivative and less than 5% docosahexaenoic acid or a docosahexaenoic acid derivative and less than 5% linoleic acid or a linoleic acid derivative.

3. Pharmaceutical formulations according to claim 1 in which the fatty acid preparation contains more than 90% eicosapentaenoic acid or eicosapentaenoic acid derivative and less than 5% docosahexaenoic acid or a docosahexaenoic acid derivative and less than 5% linoleic acid or a linoleic acid derivative.

4. Pharmaceutical formulations according to claim 1 in which the fatty acid preparation contains more than 90% eicosapentaenoic acid or eicosapentaenoic acid derivative and less than 1% docosahexaenoic acid or a docosahexaenoic acid derivative and less than 1% linoleic acid or a linoleic acid derivative.

5. Pharmaceutical formulations according to claim 1 in which the fatty acid preparation contains more than 95% eicosapentaenoic acid or eicosapentaenoic acid derivative and less than 1% docosahexaenoic acid or a docosahexaenoic acid derivative and less than 1% linoleic acid or a linoleic acid derivative.

6. Formulations according to claim 1 in which the fatty acid preparation is in the form selected from the group consisting of the free acid and a derivative selected from the group consisting of: salts such as sodium, potassium or lithium salts; esters such as ethyl esters and cholesterol esters; mono-, di- and triglycerides; amides; phospolipids; and any other derivatives able to raise the levels of the fatty acid in the blood or tissues.

7. Pharmaceutical formulations according to claim 1 in which the EPA is replaced by or added to by any one or more of preparations selected from the group consisting of: preparations of gamma-linolenic acid (GLA), preparations of dihomogamma-linolenic acid (DGLA), preparations of arachidonic acid (AA) and preparations of stearidonic acid (SA), each containing less than 10% docosahexaenoic acid and less than 10% linoleic acid.

8. Pharmaceutical formulations according to claim 1 in which the enzyme inhibitor is selected from the group consisting of a combined inhibitor of COX-1 and COX-2; a selective COX-2 inhibitor; an inhibitor of one of the LOX groups of enzymes; and a combined inhibitor of both COX and LOX enzymes.

9. Pharmaceutical formulations according to claim 1 when used for the treatment of any form of cancer or cancer cachexia.

10. Pharmaceutical formulations according to claim 1 when used for the treatment of any form of psychiatric disease including schizophrenia, schizoaffective disorders, schizotypy, depression, anxiety, bipolar disorder, mania, borderline personality disorder, alcoholism and attention deficit hyperactivity disorder or any other psychiatric illness.

11. Pharmaceutical formulations according to claim 1 when used for the treatment of any form of neurological or neurodegenerative disease including Parkinson's disease, Alzheimer's disease, Huntington's disease, amyotrophic lateral sclerosis or any other “triplet repeat” disease, stroke, multi-infarct or any other form of dementia, multiple sclerosis, chronic fatigue and epilepsy.

12. Pharmaceutical formulations according to claim 1 when used for treating any form of inflammatory disease including any form of arthritis, any form of inflammatory skin disease including psoriasis and eczema, asthma, any form of inflammatory gastrointestinal disease including ulcerative colitis and Crohn's disease, and any inflammatory conditions of any other organs including the eyes and brain.

13. Pharmaceutical formulations according to claim 1 when used for treating any form of cardiovascular or cerebrovascular disease.

14. Pharmaceutical formulations according to claim 1 when used for treating any form of respiratory disease, such as asthma or chronic obstructive pulmonary disease.

15. Pharmaceutical formulations according to claim 1 when used for treating any form of metabolic disease including diabetes, syndrome X, and any disturbance of calcium metabolism including osteoporosis, ectopic calcification or urinary tract stone formation.

16. Pharmaceutical formulations according to claim 1 when used for treating any renal or urinary tract disease.

17. Pharmaceutical formulations according to claim 1 when used for treating any form of disease of the reproductive system, including breast pain, premenstrual syndrome, dysmenorrherea or endometriosis.

18. The co-administration of a fatty acid preparation as described in the pharmaceutical formulations of claim 1 with an inhibitor of COX-1 or COX-2 or LOX or one or more of the FACL enzymes in the treatment of any of the following diseases or disorders:
any form of cancer;
any form of psychiatric disease including schizophrenia, schizoaffective disorders, schizotypy, depression, anxiety, bipolar disorder, mania, borderline personality disorder, alcoholism and attention deficit hyperactivity disorder or any other psychiatric illness;
any form of neurological or neurodegenerative disease including Parkinson's disease, Alzheimer's disease, Huntington's disease, amyotrophic lateral sclerosis or any other “triplet repeat” disease, stroke, multi-infarct or other form of dementia, multiple sclerosis, chronic fatigue and epilepsy;
any form of inflammatory disease including any form of arthritis, any form of inflammatory skin disease including psoriasis and eczema, asthma, any form of inflammatory gastrointestinal disease including ulcerative colitis and Crohn's disease, and any inflammatory conditions of any other organs including the eyes and brain;
any form of cardiovascular or cerebrovascular disease;
any form of respitory disease;
any form of metabolic disease including diabetes, syndrome X, and any disturbance of calcium metabolism including osteoporosis, unolithiase, or urinary tract stone formation;
any form of renal or urinary tract disease;
any form of disease or disorder of the reproductive system or menstrual cycle.
